(19) United States
(12) Patent Application Publication
(10) Pub. No.: US 2026/0114747 A1
(43) Pub. Date: Apr. 30, 2026

(54) DEVICES AND METHODS FOR CONTINUOUS RESPIRATORY RATE MONITORING AND VISUALIZATION

(71) Applicant: Spire, Inc., San Francisco, CA (US)

(72) Inventors: Mark HOLT, San Francisco, CA (US); Shena PARK, San Francisco, CA (US)

(21) Appl. No.: 19/009,055

(22) Filed: Jan. 3, 2025

Related U.S. Application Data
(63) Continuation of application No. PCT/US2024/040297, filed on Jul. 31, 2024.
(60) Provisional application No. 63/518,068, filed on Aug. 7, 2023.

(30) Foreign Application Priority Data
Feb. 21, 2024 (JP) 2024-024400

(51) Int. Cl. A61B 5/08; A61B 5/00

(52) U.S. Cl. CPC A61B 5/0816; A61B 5/6802

(57) ABSTRACT

A respiration monitoring system can include a respiration sensor configured to continuously monitor respiration data of a user. The respiration sensor can be attached to the user's article of clothing and be positioned between the article of clothing and a body of the user. The respiration monitoring system can include a computer readable medium storing instructions that, when executed by one or more processors, cause the one or more processors to determine a respiratory rate of the user using the respiration data, determine a respiratory rate distribution in a first respiratory rate band and a second respiratory rate band over a duration of time, and cause display of the respiratory rate distribution. The respiratory rate distribution can indicate, over the duration of time, relative proportions of time the respiratory rate falls into the first or second respiratory rate bands.

### CROSS-REFERENCE TO PRIORITY APPLICATIONS

This application is a continuation of International Patent Application No. PCT/US2024/040297, filed on Jul. 31, 2024, which claims priority to U.S. Provisional Patent Application No. 63/518,068, filed on Aug. 7, 2023 and Japanese Patent Application No. 2024-024400, filed on Feb. 21, 2024, each of which is incorporated by reference in its entirety.

### TECHNICAL FIELD

Disclosed systems and methods relate to physiological signals monitoring and, in particular, to continuous respiratory monitoring and visualization.

### BACKGROUND

Respiration parameters can provide profound insight into an individual's wellbeing. Respiration parameters can be indicative of physiological or mental states of an individual, as well as prognostic with regard to diagnosis of medical conditions. In examples, respiration parameters and other physiological parameters can provide insight into an individual's stress levels and can be used to detect more serious pulmonary disorders, such as disorders associated with chronic obstructive pulmonary disease (COPD). Traditionally, however, respiration and other biometric parameter monitoring has occurred in a clinical setting, contributing to the development of respiration monitoring devices that are motion-limiting, lack portability, are not easily integrated into the daily lives of a user, are difficult to use, or provide incomplete or inadequate information. Disclosed systems and methods solve at least these problems.

### SUMMARY

A respiration monitoring system can include a respiration sensor configured to continuously monitor a respiration data of a user. The system can include a non-transitory computer readable storage medium storing instructions that, when executed by one or more processors, cause the one or more processors to determine a respiratory rate of the user using the respiration data. The instructions can cause the one or more processors to, using the respiratory rate, determine a first respiratory rate distribution in a first respiratory rate band and a second respiratory rate band over a duration of time. The first respiratory rate distribution can indicate, over the duration of time, relative proportions of time the respiratory rate falls into the first or second respiratory rate bands. The instructions can cause the one or more processors to cause display of the first respiratory rate distribution.

The respiration monitoring system of any of the preceding paragraphs and/or any of the respiration monitoring systems disclosed herein can include one or more of the following features. The first respiratory rate distribution can be displayed as a plurality of bars corresponding to a plurality of portions of the duration of time, each bar indicating relative proportions of time the respiratory rate, over a portion of the duration of time, falls into the first or second respiratory rate bands. Relative proportions of time of the respiratory rate falling into the first or second respiratory rate bands can be illustrated in different colors.

The respiration monitoring system of any of the preceding paragraphs and/or any of the respiration monitoring systems disclosed herein can include one or more of the following features. The instructions can cause the one or more processors to, using the respiratory rate, determine the first respiratory rate distribution in the first respiratory rate band, the second respiratory rate band, and at least one additional respiratory rate band over the duration of time. The first respiratory rate distribution can indicate, over the duration of time, relative proportions of time the respiratory rate falls into the first, second, or at least one additional respiratory rate bands. Relative proportions of time of the respiratory rate falling into the first, second, or at least one additional respiratory rate bands can be illustrated in different colors.

The respiration monitoring system of any of the preceding paragraphs and/or any of the respiration monitoring systems disclosed herein can include one or more of the following features. The first respiratory rate distribution can be displayed using a rolling time window. The rolling time window can include a plurality of increments that are less than twenty-four hours. The rolling time window can include twenty-four hours and each increment of the plurality of increments can include four hours.

The respiration monitoring system of any of the preceding paragraphs and/or any of the respiration monitoring systems disclosed herein can include one or more of the following features. The instructions can cause the one or more processors to, using the respiratory rate, determine a second respiratory rate distribution in a third respiratory rate band and a fourth respiratory rate band different from the first and second respiratory rate bands over the duration of time. The second respiratory rate distribution can indicate, over the duration of time, relative proportions of time the respiratory rate falls into the third or fourth respiratory rate bands. The instructions can cause the one or more processors to display the first and second respiratory rate distributions.

The respiration monitoring system of any of the preceding paragraphs and/or any of the respiration monitoring systems disclosed herein can include one or more of the following features. The first and second respiratory rate bands can be configured to at least one of be adjustable by a healthcare provider or be dynamically adjusted responsive to changes in the respiratory rate over at least a portion of the duration of time. The respiration sensor can be configured to be attached to an article of clothing worn by a user and be positioned between the article of clothing of worn by the user and a body of the user.

Disclosed are methods of operating the respiration monitoring system of any of the preceding paragraphs and/or any of the respiration monitoring systems disclosed herein.

Disclosed are non-transitory computer readable media storing instructions that, when executed by one or more processors, cause the one or more processors to perform the methods of any of the preceding paragraphs and/or any of the methods disclosed herein.

### BRIEF DESCRIPTION OF DRAWINGS

Throughout the drawings, reference numbers may be re-used to indicate correspondence between referenced elements. The drawings are provided to illustrate example implementations described herein and are not intended to limit the scope of the disclosure.

FIG. 1 illustrates a distributed physiological monitoring system.

FIG. 2 illustrates an exploded view of a sensor for physiological monitoring.

FIGS. 3 and 4A-4C illustrate example respiratory rate plots.

### DETAILED DESCRIPTION

### Respiration Monitoring System

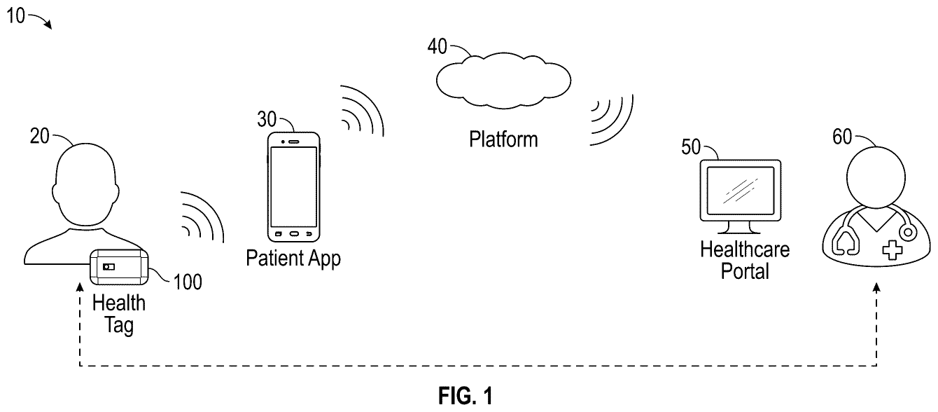

FIG. 1 illustrates a distributed physiological monitoring system 10. The system 10 can monitor one or more physiological parameters of a user 20, such as the user's respiration. While certain examples describe monitoring and processing respiration, other physiological parameters can be similarly monitored and processed.

The system 10 can continuously monitor or indicate physiological or mental states of the user 20 based upon the user's physiological characteristics (such as, respiration characteristics), and can additionally or alternatively function to facilitate diagnosis of medical conditions with cardio-pulmonary indications. In some examples, the system 10 can facilitate monitoring of the user's well-being based on respiration characteristics, including stress levels or physical exertion (such as, during exercise). For instance, the system 10 can function to indicate signs of medical conditions, such as conditions associated with COPD (for instance, apnea, asthma, or emphysema), pneumonia or lung infection (for instance, being caused by COVID-19 infection), heart disease, substance use disorder, or the like. For example, the system 10 can indicate a breathing abnormality of the user 20, which can be used to initiate diagnosis or treatment of a condition of the user's upper respiratory tract, trachea, bronchi, bronchioles, alveoli, pleura, pleural cavity, or any nerves or muscles associated with breathing. The system 10 can be configured to monitor any other suitable respiration behavior of the user 20 or indicate any other suitable sign of a respiration-related medical condition.

The system 10 can include a respiration sensor 100 that can be worn by the user 20. The respiration sensor 100 may not inhibit the mobility of the user 20, such that respiration monitoring can occur as the user performs normal activities in the user's daily life, such as, walking, exercising, working, resting, sleeping, etc. The respiration sensor 100 can include a housing that supports or encloses various components, as described herein. The housing of the respiration sensor 100 can be attached to an interior surface of a garment being worn by the user (such as, a waistband, bra, shirt, belt, etc.). The respiration sensor 100 can be positioned between the garment and the user's body. In some cases, multiple respiration sensors 100 can be coupled to a single garment of a set of garments worn by the user 20. In some instances, multiple respiration sensors 10 can be coupled to various garments that the user can choose to wear at any given time.

The respiration sensor 100 can continuously monitor the user's breathing. The respiration sensor 100 can include a housing that supports or encloses electronic circuitry configured to monitor the user's breathing. The electronic circuitry can include one or more sensors configured to deflect in response to the movements of the user's body produced by the user's breathing or to detect force in response to the user's breathing in any other suitable manner (such as, without deflection of an element of a sensor). The sensor can be directly or indirectly (such as, via the housing) robustly connection with the user 20, such that deflections associated with the user's breathing are detected with minimal interference due to other motions of the user or the respiration sensor 100. The sensor can be constrained at opposing sides, in order to enhance an effect of respiration-induced motion of the user's body (such as, abdominal region or chest region). For example, the respiration sensor 100 can be positioned between the user's clothing and the user's skin, coupled robustly to an article of the user's clothing using any one or more of an adhesive (such as, permanent adhesive or non-permanent adhesive), strap, clip, or the like.

The sensor can include a capacitor that is configured to produce an electrical signal (such as, a change in the electric field) in response to a deflection of a first capacitor plate relative to a second capacitor plate. The first and the second plates of the capacitor can be separated by a non-rigid material that provides support to each plate of the capacitor. The first and the second plates of the capacitor can be oriented with faces parallel to the surface of the user 20, such that deflections perpendicular to the faces of the capacitor plates, produced by the user's breathing, are detected by the respiration sensor 100. As such, the capacitor plates can be positioned between the user's clothing and the user's body, such as, at an abdomen region, at a chest region, between an undergarment and the user's body, etc. Deflections of one capacitor plate relative to another capacitor plate, with deflection components perpendicular to the surface of the user's body, can be detected. As such, the respiration sensor 100 may be able to produce electrical signals in response to the compression/decompression of the sensor resulting from the user's breathing. The sensor can include multiple capacitors or other sensing elements, which can facilitate determination of additional aspects of an applied force (such as, centroid of force, etc.), for example, in applications involving placement guidance for the respiration sensor 100.

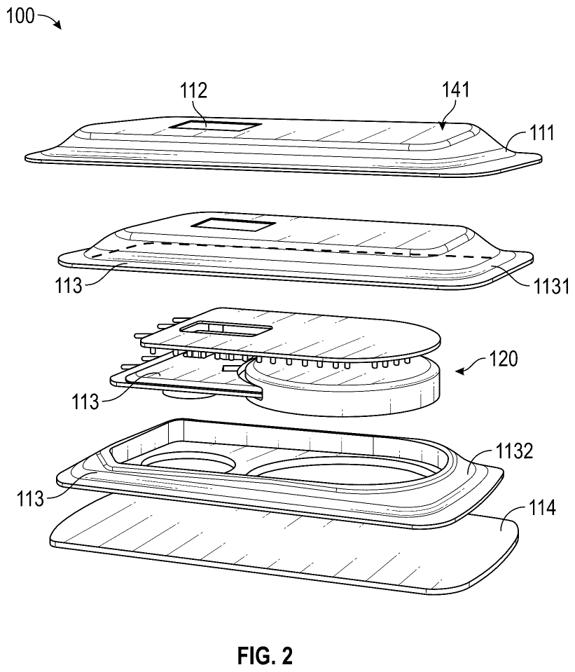

With reference to FIG. 2, the respiration sensor 100 can include electronic circuitry 120 enclosed or supported by a housing. The outer layer 111 of the housing can function to shield portions of the respiration sensor 100 from contact with the external environment. At least a portion of the outer layer can be manufactured from soft materials (such as, woven fabrics, non-woven fabrics) to provide a comfortable, non-irritating interface to the user 20. Accordingly, the outer layer 111 can function to provide a user-coupling interface 141 to the respiration sensor 100 that is comfortable for the wearer of the garment to which the respiration sensor is coupled. The outer layer can define at least one opening 112 to access the external environment. This opening can be used for sensing one or more additional physiological parameters of the user 20 or one or more parameters of the external environment.

A support structure 113 of the housing can function to resiliently retain the components of the respiration sensor 100 enclosed by the housing to enable operation of the respiration sensor and prevent breakage. The support structure 113 can include a resilient layer 1131 (such as, a thermally-formed semi-rigid plastic layer) that defines an opening, similar in size, shape, and location to the opening 112 defined in variations of the outer layer. The opening in the support structure can, in a first example, provide a path between the electronic circuitry 120 (such as, a portion of the electronics module wherein a sensor is located) and the external environment (such as, by way of a corresponding opening in the outer layer).

The support structure 113 can include a shell 1132 defining one or more voids, configured to receive and retain the electronic circuitry 120. The shell 1132 can be adjacent to the attachment module 114 (such as, at a surface of the substrate opposing the attachment layer or adhesive layer), but can, in variations, be separated from the attachment module by one or more layers or other respiration sensor components. The shell 1132 can be made of a resilient material, such as silicone, plastic (such as, PVC, ABS, HDPE, etc.), high density foam, and the like, but can additionally or alternatively be made up of any suitable material. For example, the shell 1132 can define an oblong ellipsoidal depression arranged to receive the electronic circuitry 120. As another example, the shell 1132 can define a bevel about its outer edge that defines the volumetric shape of the respiration sensor and is curved such that the external shape minimizes sharp edges or other stark morphological features that could cause user discomfort. The shell 1132 can be formed of multiple components or pieces, wherein a first piece (such as, piece 1132) defines the one or more voids for receiving components and a second piece defines the external shape and is adapted to couple to the first piece in a stacked configuration (such as, piece 1131).

The support structure 113 can include foam filler material that occupies portions of the interior volume defined by the housing that are not otherwise occupied by respiration sensor components. The foam filler material can, in variations, provide thermal insulation to passively manage heat transfer among the components of the respiration sensor 100.

The attachment module 114 can function to couple the respiration sensor 100 to a wearable article, and to cooperatively enclose components of the respiration sensor in combination with the outer layer of the housing. The attachment module 114 can include a substrate and an attachment layer. The substrate can be a resilient material (such as, a polycarbonate substrate), but can additionally or alternatively be any suitable material. The substrate can define a broad surface, at which the attachment layer is arranged. The attachment layer can be an adhesive layer, but in alternative variations the attachment layer can include any suitable mechanism for attachment to a wearable article (such as, one side of a hook-and-loop fastener, one or more buttons, a zipper, etc.). For example, the adhesive layer can include a two-part silicone elastomer adhesive.

The adhesive can cover the entirety of at least one side of the attachment module 114, but can additionally or alternatively cover any suitable portion of the attachment module or other portions of the housing, in whole or in part. In some implementations, the respiration sensor 100 can include an attachment module arranged at opposing sides of the housing, such that the respiration sensor can be attached to a garment at either of the two opposing sides.

The system 10 can include a patient application 30 (or patient app) running on a computing device, such as a mobile phone, smart watch, tablet, personal computer, custom hardware, or the like. The patient app 30 can communicate with the respiration sensor 100 and obtain user's physiological data (such as, respiratory data) collected by the respiration sensor. The communication can be wireless, such as via Bluetooth or Bluetooth Low Energy (BLE). The patient app 30 can process the physiological data, display or otherwise provide the data to the user 20, display or otherwise provide one or more instructions or alerts, etc. The patient app 30 can configure the respiration sensor 100.

The system 10 can include a processing platform 40 running on one or more computing devices, which can be one or more cloud servers. The processing platform 40 can communicate with the patient app 30 and receive user's physiological data (such as, respiratory data collected by the respiration sensor 100). The communication can be wireless, such as via a wireless area network, cellular, or the like. The processing platform 40 can execute one or more processes to analyze the user's physiological data, determine patterns, identify one or more medical conditions (such as, one or more disorders), or generate one or more alerts.

The system 10 can include a healthcare portal 50 running on one or more computing devices. The healthcare portal 50 can communicate with the processing platform 40 and the patient app 30 (or with the respiration sensor 100). The communication can be wireless, such as via a wireless area network, cellular, or the like. The processing platform 40 can provide to the healthcare portal 50 or the patient app 30 one or more analyses of the physiological data, patterns, identified medical conditions, or alerts. The healthcare portal 50 can be accessed by one or more healthcare providers 60. The healthcare portal 50 can analyze the physiological data of the user 20 and display that data or analysis to the healthcare provider 60. The healthcare portal 50 can communicate with one or more computing devices of the one or more healthcare providers 60. The healthcare portal 50 can be a web-based portal.

### Respiratory Rate Monitoring and Visualization

Respiratory rate (or breath rate) continuously determined by the system 10 using respiratory data continuously monitored by the respiration sensor 100 can be provided to the one or more healthcare providers 60 or the user 20. Respiratory rate and changes in the respiratory rate can be provided via the healthcare portal 50 or the patient app 30. Based on the respiratory rate and changes in the respiratory rate over time, a healthcare provider 60 can assess the user's well-being, diagnose one or more medical conditions, and determine one or more remedial actions.

Respiratory rate is highly regulated by the body to ensure that the rate of oxygen inflow and carbon dioxide outflow meet physiological needs. For a typical, healthy adult the normal respiratory rate at rest is 12 to 18 breaths per minute (bpm). For an adult, respiratory rate over 25 breaths per minute (and particularly over 30 breaths per minute) can correspond to an elevated respiratory rate and may indicate presence of a one or more medical conditions.

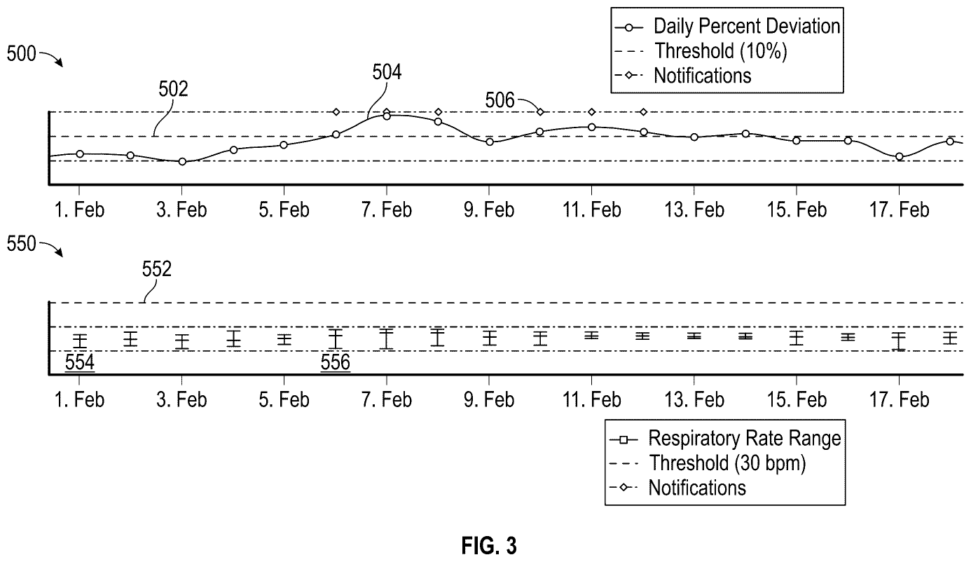

FIG. 3 illustrates graphs 500 and 550 showing changes in the respiratory rate over time. Graph 500 can illustrate a percentage deviation of the respiratory rate from a baseline. The x-axis can correspond to time (indicated in days), and the y-axis can correspond to percentage deviation from the baseline. The baseline can be an N-day average of the respiratory date, where N is an integer (such as, 1, 2, 3, etc.). In the example graph 500 illustrated in FIG. 3, the baseline can be a 90-day period. Plot 504 can illustrate daily deviation from the baseline. Threshold 502 can illustrate a 10% deviation from the baseline. Graph 500 can also illustrate notifications or alerts 506 generated when the respiratory rate on a particular day exceeds the threshold 502.

Graph 550 can illustrate respiratory rate range over time. The x-axis can correspond to time (indicated in days), and the y-axis can correspond to the number of breaths per minute. Graph 550 can illustrate a threshold 552 (for instance, 30 bpm). For a particular day, graph 550 can illustrate a respiratory rate range (such as, 554 or 556), which can include maximum, median, and minimum respiratory rates on that day.

While graphs 500 and 550 visually display respiratory rate changes over time, they may not provide full context for the respiratory rate changes, thereby reducing their usefulness. For example, graphs 500 and 550 do not provide distribution of the respiratory rate over a period of time (such as, per day). Small changes in the respiratory rate from day to day may not be visible in these graphs due to the way they aggregate data from an entire day. For example, if a user had a longer period of rapid breathing but this was offset by more time spent sleeping, when respiratory rate is naturally slower, a change would not be visible. As a result, the efficiency and effectiveness of the system 10 may be limited.

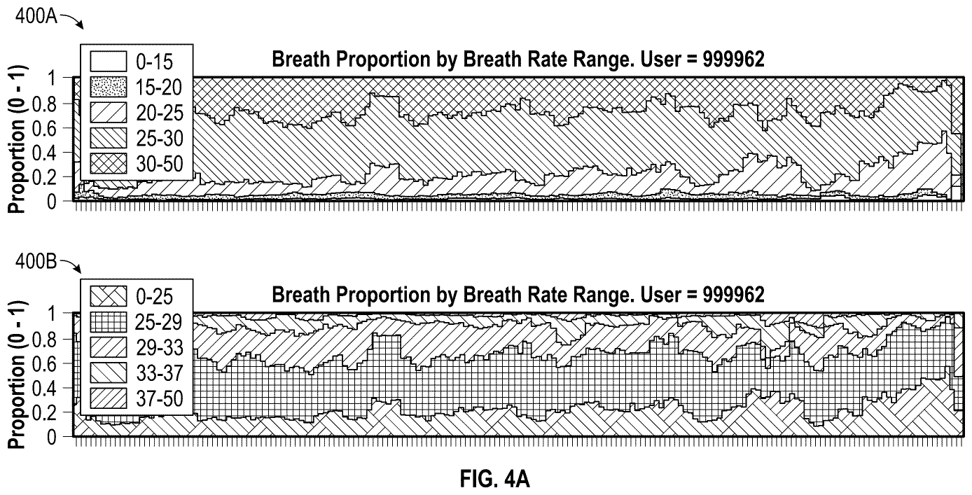

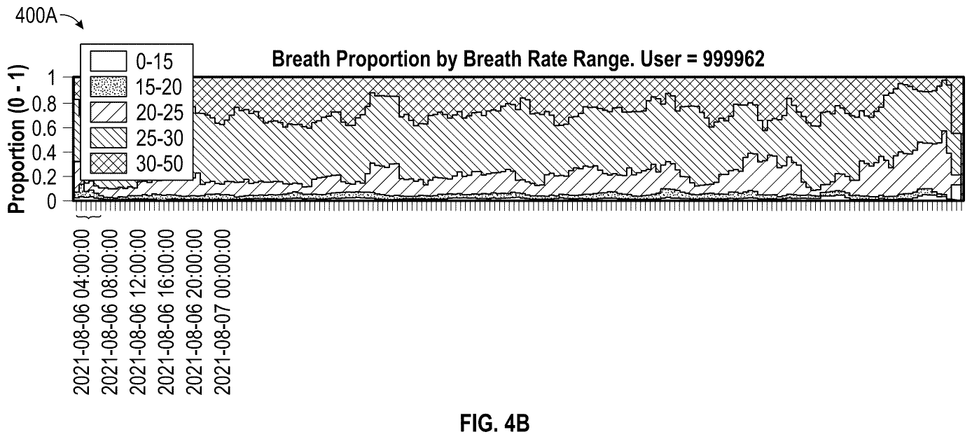

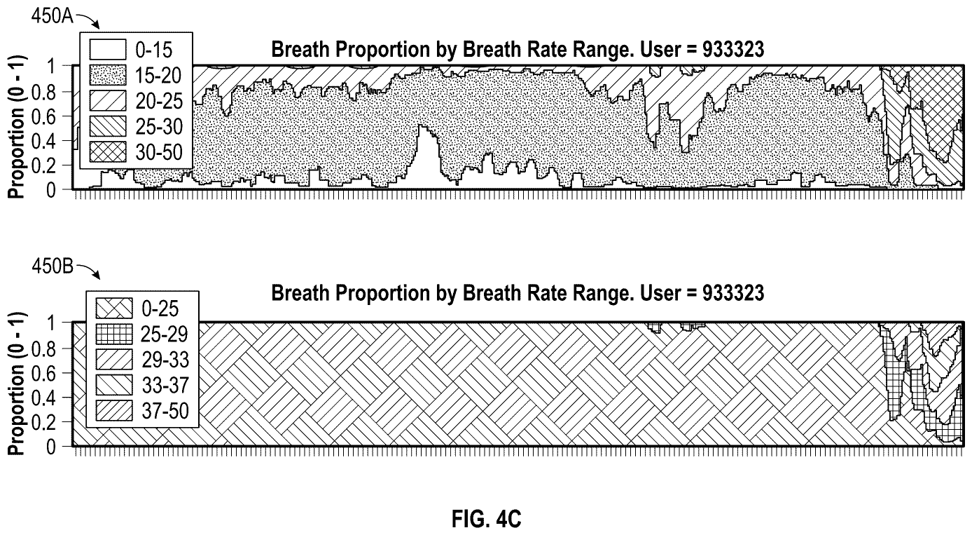

FIGS. 4A to 4C illustrate graphs showing respiratory rate distribution over time, which address the foregoing shortcomings. Graphs 400A and 400B of FIG. 4A illustrate respiratory rate distribution over a duration of time (such as, 24 hours). The y-axis can correspond to a proportion (between 0 and 1) or a percentage (between 0% and 100%), and the x-axis can correspond to a particular duration of time. The vertical bars illustrate a proportion of time over the duration of time that a user's respiratory rate falls into at least two ranges or bands (such as, normal respiratory rate and abnormal respiratory rate bands). For example, with reference to FIGS. 4A and 4B, the first vertical bar labeled “2021-08-06 04:00:00” illustrates that the user's respiratory rate over the duration of time was mostly in the band exceeding 20 breaths per minute. In fact, the majority of time, the user's respiratory rate has exceeded 25 breaths per minute, which can be indicative of the presence of one or more medical conditions. Quick examination of the graph 400A can reveal that the user is quite sick with one or more underlying medical conditions.

To provide a rich and nuanced view of the respiratory rate, the graph 400A can show distribution of the respiratory rate into five respiratory rate bands, such as, 0-15, 15-20, 20-25, 25-30, and 30-50 breaths per minute. In some implementations, less or more bands can be utilized. In other implementations, different ranges of breaths per minute could be selected for the bands, such as 0-10, 10-20, 20-25, 25-30, and 30-50 breaths per minute. Different colors can be used to indicate the proportion of time the respiratory rate has satisfied the particular band. For instance, dark green color can be associated with the band of 0-15 breaths per minute, light green color can be associated with the band of 15-20 breaths per minute, light red color can be associated with the band of 20-25 breaths per minute, red color can be associated with the band of 25-30 breaths per minute, and dark red color can be associated with the band of 30-50 breaths per minute. One or more other alternative or additional colors can be used in certain implementations. Patterns or other visual treatments could also be used in addition to, or in lieu of color, to accommodate visibility for those with color blindness or in displays where color is limited.

The duration of time for each of the vertical bars can correspond to a 24-hour time period. The use of a twenty-four hour time period can smooth out any inconsistencies that may be caused by lack of compliance resulting from the user 20 not wearing the respiration sensor 10. In some instances, the time period (such as, 24-hour time period) can be displayed in a rolling fashion to enable responsiveness in the data display without introducing excess noise. With reference to FIG. 4B, in the graph 400A, each vertical bar can represent a rolling 24-hour window, incremented every 4 hours. For example, the first vertical bar can correspond to a distribution of the user's respiratory rate over 24-hours (2021-08-05 4 am to 2021-08-06 4 am). As another example, the second vertical bar can correspond to a distribution of the user's respiratory rate over the 24-hours leading to 8 am (2021-08-05 8 am to 2021-08-06 8 am). As yet another example, the third vertical bar can correspond to a distribution of the user's respiratory rate over the 24-hours leading to 12 pm (2021-08-05 12 pm to 2021-08-06 12 pm). As yet another example, the fourth vertical bar can correspond to a distribution of the user's respiratory rate over the 24-hours leading to 4 pm (2021-08-05 4 pm to 2021-08-06 4 pm). As yet another example, the fifth vertical bar can correspond to a distribution of the user's respiratory rate over the 24-hours leading to 8 pm (2021-08-05 8 pm to 2021-08-06 8 pm). As yet another example, the sixth vertical bar can correspond to a distribution of the user's respiratory rate over the 24-hours leading to 12 am (2021-08-06 12 am to 2021-08-07 12 am).

The use of such rolling windows of X-hours can advantageously facilitate responsiveness to respiratory rate changes. For instance, if the user's breath accelerates in an X-hour period, this would be captured by the graph 400A. While 24-hour rolling windows are utilized in the graph 400A, windows can be shortened or lengthened in some implementations and the increment of time that each window advances can also be shortened or lengthened. In some cases, shortening the window or time increment may clutter the graph 400A with unnecessary detail, while lengthening the window or time increment can cause being less responsive to respiratory rate changes.

With reference to FIG. 4A, the graph 400B can illustrate the same respiratory data as shown in the graph 400B, but using different respiratory rate bands. As is shown, higher respiratory rate bands are utilized in the graph 400B, such as, 0-25 breaths per minute, 25-29 breaths per minute, 29-33 breaths per minute, 33-37 breaths per minute, and 37-50 breaths per minute. Such change in the respiratory band scale can provide a more nuanced view of the respiratory rate over time, particularly for the illustrated user whose respiratory rate is outside the normal range. For case of interpretation, the graph 400B can use a different color-coding scheme than the graph 400A. For instance, dark blue color can be associated with the band of 0-25 breaths per minute, light blue color can be associated with the band of 25-29 breaths per minute, light brown color can be associated with the band of 29-33 breaths per minute, brown color can be associated with the band of 33-37 breaths per minute, and dark brown color can be associated with the band of 37-50 breaths per minute.

In some cases, the graph 400A can serve as the primary view of the respiratory rate distribution over time, and the graph 400B can serve as the secondary view of the respiratory rate distribution over time. In other implementations, a tertiary view with an alternate range could also be introduced.

In some cases, the respiratory rate bands in the graphs 400A or 400B can be defined or changed by the healthcare provider 60 or the user 20. For instance, the number of different bands and the ranges of the different bands can be selected. In some instances, the number of bands and the ranges of the bands can be changed by the system 10 dynamically responsive to the changes in the respiration data. For instance, the ranges of the different bands in the secondary view can be adjusted based on whether the respiratory rate of the user is normal, elevated, or highly elevated.

FIG. 4C illustrates respiratory rate distribution over time for a different user than that of FIGS. 4A and 4B. Similarly to FIG. 4A, primary and secondary graphs 450A and 450B are illustrated. In comparison with the graphs in FIGS. 4A and 4B, quick examination of the graphs in FIG. 4C can reveal that the user whose respiratory rate distribution is illustrated in FIG. 4C has a much lower respiratory rate. In fact, for the majority of time period illustrated in FIG. 4C, the user's respiratory rate does not exceed 25 breaths per minute. However, it is readily apparent that the user's respiratory rate has increased in in the latter part of the time period, which may be indicative of an onset of one or more underlying medical conditions.

In some implementations, one or more alerts can be automatically generated by the system 10. For instance, an alert can be generated responsive to determining that the respiratory rate distribution over a period of time satisfies a threshold proportion of time in a certain respiratory rate band. For example, an alert can be generated responsive to determining that the respiratory rate distribution over one or more 24-hour periods exceeds 25 breaths per minute at least 25% of the time.

In some implementations, the respiratory rate distribution over time could be displayed such that the data can be viewed in a scrolling fashion, advancing backwards or forwards in time. In other implementations, the respiratory rate distribution over time may be displayed over a fixed period of time that may be selected by the user or automatically selected by system 10. For instance, such a fixed time period may be used in a printed report.

In some cases, the respiratory rate distribution could be visualized with horizontal bars instead of vertical bars. Here, each horizontal bar can include multiple bands of respiratory rate and time can advance vertically to the next horizontal bar. Such a display may provide the capability of scrolling vertically through time. This approach may be more conducive to display in certain formats, for example, in mobile or tablet screens where space is more limited.

In some implementations, respiratory rate visualization may be separated not just by time, but by the activity or state of the user. For instance, a user's respiratory rate while sleeping, while at rest or while active, could be displayed separately in a unique respiratory rate distribution graph for each state. Such a display can enable easier comparisons of the user's respiratory rate, with less variation arising from the composition of respective activities. In some cases, the respiratory rates corresponding to different states of the user could be displayed in a combined stacked graph or with alternating bars, differentiated by spacing, patterning or alternate colors.

Disclosed approaches improve the efficiency of using a continuous respiration monitoring system by, for instance, providing rich, detailed, and easy-to-understand visualization of the respiratory rate distribution over time. Disclosed approaches allow a healthcare provider to quickly interpret respiratory data of interest without taking any additional actions. This can obviate the need for the healthcare provider to perform additional monitoring or analysis of the respiratory rate to assess a patient's health and, if appropriate, recommend one or more remedial actions. As a result, the respiration monitoring system can function more efficiently and effectively.

### Other Variations

While certain examples relate to monitoring respiration and providing indication of the respiratory rate, other physiological parameters can be similarly monitored and one or more visualizations of changes over time can be similarly provided. For example, activity levels, resting heart rate, movement in sleep, or the like can be monitored and one or more visualizations of changes over time can be provided.

The design may vary as components may be added, removed, or modified. Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The user interface screens or components illustrated and described herein are merely illustrative examples and can be varied in other embodiments. For instance, bars, legends, lines, buttons, dropdown boxes, select boxes, text boxes, check boxes, slider controls, or other user interface components shown may be substituted with other types of user interface components that provide the same or similar functionality. Further, the user interface components may be combined or divided into other sets of user interface components such that similar functionality, or the same functionality may be provided with very different looking user interfaces. Moreover, each of the user interface components may be selected by a user using one or more input options, such as a mouse, touch screen input, keyboard input, or voice input, among other user interface input options.

User interface screens illustrated and described herein can include additional or alternative components. These components can include menus, lists, buttons, text boxes, labels, radio buttons, scroll bars, sliders, checkboxes, combo boxes, status bars, dialog boxes, windows, and the like. User interface screens can include additional or alternative information. Components can be arranged, grouped, displayed in any suitable order.

The various illustrative logical blocks, modules, routines, and algorithm steps described in connection with the embodiments disclosed herein can be implemented as electronic hardware, or combinations of electronic hardware and computer software. To clearly illustrate this interchangeability of hardware and software, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor device can be a microprocessor, but in the alternative, the processor device can be a controller, microcontroller, or state machine, combinations of the same, or the like. A processor device can include electronic circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, or within less than 0.01% of the stated amount.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A respiration monitoring system comprising:
a respiration sensor configured to continuously monitor a respiration data of a user, the respiration sensor configured to be attached to an article of clothing worn by the user and be positioned between the article and a body of the user; and
a non-transitory computer readable storage medium storing instructions that, when executed by one or more processors, cause the one or more processors to:
determine a respiratory rate of the user using the respiration data;
using the respiratory rate, determine a first respiratory rate distribution in a first respiratory rate band and a second respiratory rate band over a duration of time, the first respiratory rate distribution indicating, over the duration of time, relative proportions of time the respiratory rate falls into the first or second respiratory rate bands; and
cause display of the first respiratory rate distribution.

2. The respiration monitoring system of claim 1, wherein the first respiratory rate distribution is displayed as a plurality of bars corresponding to a plurality of portions of the duration of time, each bar indicating relative proportions of time the respiratory rate, over a portion of the duration of time, falls into the first or second respiratory rate bands.

3. The respiration monitoring system of claim 2, wherein relative proportions of time of the respiratory rate falling into the first or second respiratory rate bands are illustrated in different colors.

4. The respiration monitoring system of claim 2, wherein the instructions cause the one or more processors to:
using the respiratory rate, determine the first respiratory rate distribution in the first respiratory rate band, the second respiratory rate band, and at least one additional respiratory rate band over the duration of time, the first respiratory rate distribution indicating, over the duration of time, relative proportions of time the respiratory rate falls into the first, second, or at least one additional respiratory rate bands.

5. The respiration monitoring system of claim 4, wherein relative proportions of time of the respiratory rate falling into the first, second, or at least one additional respiratory rate bands are illustrated in different colors.

6. The respiration monitoring system of claim 1, wherein the first respiratory rate distribution is displayed using a rolling time window.

7. The respiration monitoring system of claim 6, wherein the rolling time window comprises a plurality of increments that are less than twenty-four hours.

8. The respiration monitoring system of claim 7, wherein the rolling time window comprises twenty-four hours and each increment of the plurality of increments comprises four hours.

9. The respiration monitoring system of claim 1, wherein the first and second respiratory rate bands are configured to at least one of: be adjustable by a healthcare provider or be dynamically adjusted responsive to changes in the respiratory rate over at least a portion of the duration of time.

10. The respiration monitoring system of claim 1, wherein the instructions further cause the one or more processors to:
using the respiratory rate, determine a second respiratory rate distribution in a third respiratory rate band and a fourth respiratory rate band different from the first and second respiratory rate bands over the duration of time, the second respiratory rate distribution indicating, over the duration of time, relative proportions of time the respiratory rate falls into the third or fourth respiratory rate bands; and
cause display of the first and second respiratory rate distributions.

11. A non-transitory computer readable storage medium storing instructions that, when executed by one or more processors, cause the one or more processors to:
determine a respiratory rate of a user using respiration data continuously collected by a respiration sensor;
using the respiratory rate, determine a first respiratory rate distribution in a first respiratory rate band and a second respiratory rate band over a duration of time, the first respiratory rate distribution indicating, over the duration of time, relative proportions of time the respiratory rate falls into the first or second respiratory rate bands; and
cause display of the first respiratory rate distribution.

12. The non-transitory computer readable storage medium of claim 11, wherein the first respiratory rate distribution is displayed as a plurality of bars corresponding to a plurality of portions of the duration of time, each bar indicating relative proportions of time the respiratory rate, over a portion of the duration of time, falls into the first or second respiratory rate bands.

13. The non-transitory computer readable storage medium of claim 12, wherein relative proportions of time of the respiratory rate falling into the first or second respiratory rate bands are illustrated in different colors.

14. The non-transitory computer readable storage medium of claim 12, wherein the instructions cause the one or more processors to:
using the respiratory rate, determine the first respiratory rate distribution in the first respiratory rate band, the second respiratory rate band, and at least one additional respiratory rate band over the duration of time, the first respiratory rate distribution indicating, over the duration of time, relative proportions of time the respiratory rate falls into the first, second, or at least one additional respiratory rate bands.

15. The non-transitory computer readable storage medium of claim 14, wherein relative proportions of time of the respiratory rate falling into the first, second, or at least one additional respiratory rate bands are illustrated in different colors.

16. The non-transitory computer readable storage medium of claim 11, wherein the first respiratory rate distribution is displayed using a rolling time window.

17. The non-transitory computer readable storage medium of claim 16, wherein the rolling time window comprises a plurality of increments that are less than twenty-four hours.

18. The non-transitory computer readable storage medium of claim 17, wherein the rolling time window comprises twenty-four hours and each increment of the plurality of increments comprises four hours.

19. The non-transitory computer readable storage medium of claim 11, wherein the first and second respiratory rate bands are configured to at least one of: be adjustable by a healthcare provider or be dynamically adjusted responsive to changes in the respiratory rate over at least a portion of the duration of time.

20. The non-transitory computer readable storage medium of claim 11, wherein the instructions further cause the one or more processors to:
using the respiratory rate, determine a second respiratory rate distribution in a third respiratory rate band and a fourth respiratory rate band different from the first and second respiratory rate bands over the duration of time, the second respiratory rate distribution indicating, over the duration of time, relative proportions of time the respiratory rate falls into the third or fourth respiratory rate bands; and
cause display of the first and second respiratory rate distributions.
